(19) United States
(12) Patent Application Publication
(10) Pub. No.: US 2026/0109751 A1
(43) Pub. Date: Apr. 23, 2026

(54) METHODS OF DULAGLUTIDE PURIFICATION USING HYDROPHOBIC INTERACTION CHROMATOGRAPHY

(71) Applicant: ELI LILLY AND COMPANY, Indianapolis, IN (US)

(72) Inventors: Amy Lynn HUEBNER, Bridgewater, NJ (US); Darren Jerome CROWLEY, Wilton (IE); John Richard CATCHPOLE, Bishopstown (IE); Ceara de Lacy CLANCY, Belgooly (IE)

(21) Appl. No.: 19/143,256

(22) PCT Filed: Dec. 22, 2023
(86) PCT No.: PCT/US2023/085746

§ 371 Date: Jun. 25, 2025

Related U.S. Application Data
(60) Provisional application No. 63/436,365, filed on Dec. 30, 2022.

(51) Int. Cl. C07K 14/605; C07K 1/20

(52) U.S. Cl. CPC C07K 14/605; C07K 1/20; C07K 2319/30

(57) ABSTRACT

The present disclosure is directed to methods of purifying a dulaglutide composition. For example, the methods comprise loading the dulaglutide composition in-line with a buffer B solution onto a hydrophobic interaction chromatography (HIC) column in a downflow direction, washing the HIC column with a buffer mixture of a buffer A solution and the buffer B solution in the downflow direction, and eluting dulaglutide from the HIC column. A linear gradient of a percentage of the buffer A and buffer B solutions is used for eluting dulaglutide, which is then followed by an isocratic hold based on a UV absorbance reading of an eluate. The methods further comprise maintaining the isocratic hold until a front side cut is made, and then dropping the buffer B solution for dulaglutide to elute from the HIC column.

Production of recombinant Fc-containing proteins (e.g., dulaglutide) for therapeutic use typically involves the expression of the proteins in mammalian cells and subsequent purification of these proteins from host cell contaminants. Host cell contaminants include both non-proteinaceous (e.g., DNAs, RNAs, lipids, etc.) and proteinaceous components (e.g., peptides and proteins from the host cells often called host cell proteins (HCPs)). If these contaminants are not removed, they may retain their endogenous properties and degrade the product (e.g., endogenous proteinases) or impact a patient treated with the recombinant Fc-containing protein product. Additionally, the durability of purification elements, such as chromatography resins could be important in the cost and/or efficiency of an overall process. For example, choosing resins that can be cleaned and reused additional times would reduce the frequency of changing out the resin and may reduce the overall cost of a process over a number of runs. Further, methods of using chromatography resins can be optimized for efficiency.

Accordingly, there is a need for improved and/or alternative methods to select and use chromatography techniques and resins to efficiently purify Fc-containing proteins to remove host cell contaminants.

Dulaglutide (LY2189265, GLP-Fc) is a glucagon-like peptide-1 (GLP-1) analog that has been fused through a short flexible peptide linker sequence to a modified Fc portion of a human immunoglobulin G4 (IgG4). It is the active ingredient in Trulicity®, which is approved for use as an adjunct to diet and exercise to improve glycemic control in patients with type II diabetes. The dulaglutide molecule is a stand-alone therapy or is used in combination with oral agents (e.g., sulfonylurea (SU), metformin (MET), thiazolidinedione (TZD)), to improve glycemic control in patients with type II diabetes mellitus (T2DM). Dulaglutide was initially approved by the FDA in 2014, and in February 2020 was approved for use in patients with T2DM and multiple cardiovascular risk factors for the prevention of cardiovascular events. It is the first T2DM drug approved to reduce major adverse cardiovascular events (MACE) risk in primary and secondary prevention populations. Dulaglutide's structure, function, production, and use in treating T2DM are described in more detail in U.S. Pat. No. 7,452,966 and U.S. Patent Application Publication No. US 2010/0196405.

To manufacture dulaglutide, mammalian cell culture techniques are used to synthesize and secrete the dulaglutide protein. For example, the process comprises expanding a dulaglutide working cell bank of cells such as Chinese hamster ovary (CHO) cells through a series of shake flasks and seed bioreactors to generate sufficient biomass for an inoculation of a production bioreactor. In the production bioreactor, dulaglutide is secreted from the cultured CHO cells with several culturing process steps to optimize, e.g., product quality and process performance.

Once cell culturing meets certain criteria (e.g., length of culture time, titer/cell density, number of passages, number of doublings, and/or genetic stability), a primary recovery process of dulaglutide from the production process proceeds. One example of a primary recovery process comprises centrifugation for removing the CHO cells and large, insoluble, cellular debris followed by a normal flow filtration sequence for the removal of the fine, suspended particulate. The resultant filtrate from the primary recovery sequence then undergoes a detergent viral inactivation (DVI) through means of detergent added to the clarified filtrate. After the detergent inactivation, the dulaglutide is purified away from the proteinaceous and non-proteinaceous components of the product stream via a protein A affinity capture chromatographic step. The captured dulaglutide mainstream from the column then undergoes low pH viral inactivation/neutralization followed by protease heat inactivation. Following the two respective inactivation steps, anion exchange chromatography (AEX) is utilized to separate out the dulaglutide process and product-related impurities, including host cell proteins (HCP).

During several of the purification process steps, dulaglutide is measured, quantified, and identified as an IgG-GLP fusion protein. For example, during affinity capture steps, dulaglutide can be measured based on the IgG-GLP fusion protein content. For other IgG-specific quantitation assays, as well as through the inherent specificity of the downstream purity assays, dulaglutide may be identified by measuring the IgG-GLP fusion protein content. The quantity of dulaglutide may also be monitored. For example, prior to affinity capture, dulaglutide protein content is monitored by the use of a Protein A affinity capture high-performance liquid chromatography (HPLC) method. Post affinity capture chromatography step, protein purity is generally anticipated to be greater than 95% dulaglutide-related substances, and in-line spectrophotometric measurements can be employed for the dulaglutide downstream process.

The dulaglutide manufacturing process may result in one or more impurities such as Host Cell Protein (HCP), Host Cell DNA, residual Protein A (rProA), residual Triton X-100, and residual insulin. Acceptable reduction and/or clearance of HCP, DNA, and rProA may be monitored and measured at various steps of the purification process. Process performance and consistency may also be demonstrated by monitoring the reduction and/or removal of these impurities during the dulaglutide drug substance manufacturing process. The remaining process-related impurities (e.g., residual Triton X-100, residual insulin) may also be tested and measured.

Additional purification steps are utilized to further purify dulaglutide and reduce one or more impurities from the production process, e.g., after AEX discussed above. For example, additional chromatography purification by ceramic hydroxyapatite (CHT) columns can be used. The use of CHT columns, however, has a limited lifetime in the production process and can be expensive and possibly lead to downtime when CHT resins need to be replaced. As such, Applicants sought to develop additional chromatography purification methods to, e.g., increase resin lifetime and/or maintain the functional aspects of purification, i.e., increase the purity of dulaglutide and removal of impurities from the production process of dulaglutide.

### SUMMARY

The present disclosure generally relates to methods for purifying dulaglutide compositions using a hydrophobic interaction chromatography (HIC) column. Such methods comprise a loading step, a washing step, and an eluting step. The loading step comprises loading the dulaglutide composition in-line with a buffer B solution onto the HIC column in a downflow direction. The washing step comprises washing the HIC column with a buffer mixture of a buffer A and the buffer B solutions. The eluting step comprises eluting dulaglutide from the HIC column using a linear gradient of a percentage of the buffer A and buffer B solutions, which is followed by an isocratic hold based on a UV absorbance reading of an eluate from the HIC column indicating a protein-containing material is eluting from the HIC column. The methods further comprise maintaining the isocratic hold and then dropping the buffer B solution to 0% for dulaglutide to elute from the HIC column.

In some embodiments, a method for purifying dulaglutide compositions using a hydrophobic interaction chromatography (HIC) column comprises loading the dulaglutide composition in-line with a buffer B solution at a volume ratio ranging from 62.7% to 72.5% and at a linear rate ranging from 80 cm/hr-160 cm/hr onto the HIC column in a downflow direction, wherein dulaglutide from the dulaglutide composition binds to the HIC column and the composition comprises a plurality of compounds from a fermentation process of mammalian cells. The method further comprises washing the HIC column with a buffer mixture comprising from 15% to 25% of a buffer A solution and from 75% to 85% of the buffer B solution in the downflow direction at a linear rate ranging from 80 cm/hour-160 cm/hour for a duration of 2.7 column volumes (CV)-3.3 CV. The method then comprises eluting dulaglutide from the HIC column using a linear gradient comprising from 67.5% of the buffer B solution to 10% of the buffer B solution for a duration of 10 CV-12 CV, which is followed by an isocratic hold based on the UV absorbance reading of an eluate at A 280 nm with a range from 0.1 AU/cm to 0.5 AU/cm indicating a protein-containing material is eluting from the HIC column. The isocratic hold is maintained for 1.25 CV-1.75 CV, at which point a front side cut is made and mainstream collection begins with an elution gradient dropping the buffer B solution from its isocratic hold to 0%.

It is to be understood that both the foregoing general description and the following detailed description are exemplary and explanatory only and are not restrictive of the disclosure, as claimed.

The accompanying drawings, which are incorporated in and constitute a part of this specification, illustrate one (several) embodiment(s) of the disclosure and together with the description, serve to explain the principles of the present disclosure.

### BRIEF DESCRIPTION OF DRAWINGS

Exemplary embodiments are illustrated in referenced figures of the drawings. It is intended that the embodiments and figures disclosed herein are to be considered illustrative rather than restrictive.

FIG. 1 illustrates a unit operation for purifying a dulaglutide composition using a HIC column according to some embodiments herein.

FIG. 2 is a chromatogram overlay of an embodiment of the present disclosure for purifying a dulaglutide composition.

FIG. 3 is a comparative chromatogram overlay of four types of HIC column resins directed to the methods according to some embodiments herein.

### DESCRIPTION

Before the present compositions and methods are described, it is to be understood that the present disclosure is not limited to the composition comprising dulaglutide, methodologies, or protocols described, as these may vary. It is also to be understood that the terminology used in the description is for the purpose of describing the particular versions or embodiments only and is not intended to limit the scope of the present disclosure which will be limited only by the appended claims.

The terms used herein have meanings recognized and known to those of skill in the art, however, for convenience and completeness, particular terms and their meaning are set forth below.

Further, as used herein and in the appended claims, the singular forms “a”, “an”, and “the” include plural references unless the context clearly dictates otherwise. Thus, for example, reference to “sphere” is a reference to one or more spheres and equivalents thereof known to those skilled in the art, and so forth. Unless defined otherwise, all technical and scientific terms used herein have the same meanings as commonly understood by one of ordinary skill in the art. Although any methods and material similar or equivalent to those described herein can be used in the practice or testing of embodiments of the present disclosure, the preferred methods, devices, and materials are described below. All publications mentioned herein are incorporated by reference. Nothing here is to be construed as an admission that the present disclosure is not entitled to antedate such disclosures by virtues of prior disclosures.

As used herein, the term “about,” when used in connection with a specific value, means that acceptable deviations from that value are also encompassed. In certain embodiments, the term “about” means that a value higher or lower than the given value by 1%, 3%, 5%, or 10% is encompassed.

When used herein, the term “dulaglutide” refers to any GLP-1 receptor agonist protein dimer of two monomers having the amino acid sequence of SEQ ID NO:1, including any protein that is the subject of a regulatory submission seeking approval of a GLP-1 receptor agonist product which relies in whole or part upon data submitted to a regulatory agency by Eli Lilly and Company relating to dulaglutide, regardless of whether the party seeking approval of said protein actually identifies the protein as dulaglutide or uses some other term. Dulaglutide agonizes the GLP-1 receptor resulting in stimulation of insulin synthesis and secretion and has been shown to provide improved glycemic control in T2DM patients. Each monomer of dulaglutide has the amino acid sequence set forth in SEQ ID NO: 1.

(SEQ ID NO: 1)
HGEGTFTSDVSSYLEEQAAKEFIAWLVKGGGGGGGSGGGGSGGGGSAESK
YGPPCPPCPAPEAAGGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLG.

The term “mammalian cell” as used herein refers to a cell from any mammalian species including without limitation mice, rats, rabbits, dogs, primates, and, for example, humans.

As used herein, hydrophobic interaction chromatography (HIC) separates proteins based on their differences in their surface hydrophobicity by utilizing a reversible interaction between the proteins and the hydrophobic surface of the HIC resin. Generally, the binding between hydrophobic proteins and ligands of the HIC resin is affected by the presence of ion interactions of the running buffer and/or mobile phase. A high salt concentration enhances the interaction, while lowering the salt concentration weakens the interaction. As the ionic strength of the buffer solution is reduced, the interaction between the protein and the matrix/resin is reversed, and the protein with the lowest degree of hydrophobicity is eluted first. The most hydrophobic protein elutes last, requiring a greater reduction in salt concentration to reverse the interaction.

As used herein, the term “isocratic hold” refers to a period when the ratio of individual buffer solutions in the mixture of the two buffer solutions is consistent over the complete chromatography testing time.

As used herein, the term “buffer solution” may be alternatively defined as “mobile phase”, or “solvent”, or “eluent”, or “medium”. The term “buffer” refers to a solution that maintains the pH of the solution at a certain range and increases the amount of acid or alkali that must be added to cause a unit change in pH.

As used herein, the term “downflow direction” may be referred to as a flow in the reverse or opposite direction to the “upflow direction” or vice versa. Separation may be achieved by pumping a buffer solution or mobile phase upwards or against the gravity through the HIC column, or by flowing the buffer solution or mobile phase downwards or towards the gravity through the HIC column. It will be understood by the skilled person that the column may be operated in either a “downflow” mode, as described above, or in an “upflow” mode where the direction of flow of the mobile phase is reversed such that it moves up the HIC column. In a downflow mode, the buffer solution or mobile phase may enter the HIC column via the top section, move vertically along the column bed and flow downwards or towards gravity through the column bed, and finally exit the HIC column through the bottom portion. In contrast, in an upflow mode, the buffer solution or mobile phase may enter the HIC column via the bottom section, move vertically along the column bed and flow upwards or against gravity through the column bed, and finally exit the HIC column through the top portion.

As used herein, the term “eluate” refers to a liquid media comprising a molecule of interest such as dulaglutide or a protein-containing material which can be obtained subsequent to the binding of the molecule of interest to a HIC column and addition of an elution buffer solution to dissociate the molecule of interest from the HIC column.

As used herein, the term “linear gradient” refers to the technique of altering the composition of the mobile phase or the buffer solutions during a chromatography run such that the composition of the buffer mixture changes linearly over time.

As used herein, the term “loading” or alternatively “charging” refers to the amount of a molecule of interest such as dulaglutide or a protein-containing material introduced into a HIC column, expressed in grams of the molecule of interest per liter of the binding substance such as resin in the HIC column.

As used herein, the term “washing” refers to the process of releasing unbound molecules to the HIC column, such as impurities present in a loading solution to flow with the solvent or mobile phase used.

The present disclosure is directed to methods of purifying a dulaglutide composition, the method comprising: loading the dulaglutide composition in-line with a buffer B solution onto a hydrophobic interaction chromatography (HIC) column in a downflow direction, wherein dulaglutide from the dulaglutide composition binds to the HIC column; washing the HIC column with a buffer mixture of a buffer A and the buffer B solutions; and eluting dulaglutide from the HIC column using a linear gradient of a percentage of the buffer A and the buffer B solutions, wherein the linear gradient is followed by an isocratic hold based on a UV absorbance reading of an eluate from the HIC column, maintaining the isocratic hold until a front side cut is made, and then dropping the buffer B solution to 0% for dulaglutide to elute from the HIC column.

### Loading

As disclosed herein, methods of the present disclosure comprise loading the dulaglutide composition in-line with a buffer B solution onto a hydrophobic interaction chromatography (HIC) column in a downflow direction, wherein dulaglutide from the dulaglutide composition binds to the HIC column.

In some embodiments, a ratio between the dulaglutide composition and the buffer B solution when loading the dulaglutide composition in-line with a buffer B solution onto the HIC column may range from 37.5%: 62.7% to 27.5%: 72.5% of the dulaglutide composition: the buffer B solution.

In some embodiments, loading the buffer B solution with the dulaglutide composition onto the HIC column may increase the hydrophobicity of dulaglutide in the composition and ensure the binding of dulaglutide to the HIC column resin.

In some embodiments, when loading the dulaglutide composition in-line with the buffer B solution onto the HIC column, a target load may range from 10 grams-20 grams of dulaglutide per liter of HIC resin.

In some embodiments, loading of the dulaglutide composition onto the HIC column may be at a linear flow rate ranging from 80 centimeters per hour (cm/hr) to 160 cm/hr. In some embodiments, the linear rate may be 120 cm/hr.

In some embodiments, the dulaglutide composition may be a downstream product of an anionic exchange chromatography column from a manufacturing and purification process of dulaglutide. The dulaglutide composition may comprise a plurality of compounds or impurities including host cell proteins (HCPs), residual host cell proteins (rHCPs), host cell DNA (HCDNA), residual protein A (rProA), residual Triton X-100, and residual insulin.

In some embodiments, a concentration of protein-containing material in the dulaglutide composition may not be more than 7.8 grams per liter (g/L). In some embodiments, a concentration of HCP in the dulaglutide composition loaded to the HIC column may be ranging from 30 ppm to 70 ppm. A majority of the HCP removal may occur before the anionic exchange chromatography unit, with a subsequent reduction in the HCP concentration occurring during the anionic exchange chromatography unit operation. HCPs may be subsequently removed at other unit operations prior to the anionic exchange chromatography unit such as an affinity capture chromatography unit, and a low pH viral inactivation unit. Therefore, the concentration of HCPs in the dulaglutide composition loaded onto the HIC column may be insignificant compared to the dulaglutide concentration in the loaded composition.

In some embodiments, the concentration of host cell DNA (HCDNA), residual protein A (rProA), residual Triton X-100, and residual insulin in the dulaglutide composition loaded to the HIC column may be insignificant compared to the concentration of dulaglutide. The majority of the impurities-HCDNA, rProA, residual Triton X-100, and residual insulin may be removed in unit operations prior to the anionic exchange chromatography unit with subsequent removal of the HCDNA, rProA, residual Triton X-100, and residual insulin may be occurring during the anionic exchange chromatography unit operation prior to the HIC unit. In some embodiments, a trace amount of these impurities may be present in the dulaglutide composition.

### Buffer B Solution

In some embodiments, the buffer B solution may comprise a salt chosen from ammonium sulfate, sodium sulfate, sodium chloride, ammonium chloride, sodium bromide, and a combination thereof. In some embodiments, the buffer B solution comprises a sulfate salt, tris(hydroxymethyl)aminomethane (Tris), or a combination thereof. The sulfate salt may be chosen from ammonium sulfate and sodium sulfate. In some embodiments, the buffer B solution may comprise a cation chosen from Ba²⁺, Ca²⁺, Mg²⁺, Na⁺, K⁺, Rb⁺, and NH₄⁺, and/or an anion chosen from PO₄³⁻, SO₄²⁻, CH₂CO₃, Cl⁻, Br⁻, NO₃⁻, I⁻, and SCN⁻ or a combination thereof.

In some embodiments, the buffer B solution comprises Tris and sodium sulfate salt. For example, loading the dulaglutide composition in-line with the buffer B solution may comprise 20 millimolar (mM) Tris and 400 mM sodium sulfate in the buffer B solution.

In some embodiments, a conductivity of the buffer B solution may be maintained at a target range by maintaining the concentration of the buffer B solution at a target range for HIC unit operation. For example, the range of the buffer B solution concentrations from 328.9 mM to 455.2 mM may result in a ±6 millisiemens per centimeter (mS/cm) conductivity difference from the target conductivity range for the buffer B solution.

### HIC Column

As disclosed herein, the methods comprise loading dulaglutide composition in-line with the buffer B solution onto a HIC column. Hydrophobic interaction chromatography (HIC) is a purification technique that is based on the interaction of the material, such as, the resin in the HIC column with the hydrophobic region of a molecule. Depending on the partition coefficients of molecules in a medium in contact with the HIC column, the HIC column may have a preferential binding to a molecule of interest from a mixture of molecules in the medium. As disclosed in the methods herein, after loading a dulaglutide composition in-line with the buffer B solution, dulaglutide may bind to the HIC column resin.

In some embodiments, the HIC column may have a diameter ranging from 50 cm to 200 cm. For example, a 200 cm diameter HIC column may be employed for the HIC unit operation.

In some embodiments, the HIC column may have a packed bed height ranging from 15 cm to 30 cm. For example, the HIC column may have a packed bed height of 21 cm.

In some embodiments, the HIC column may comprise a material with a high mechanical strength that can sustain high pressure. For example, the HIC column may comprise a stainless-steel body.

In some embodiments, the HIC column comprises one or more upstream in-line filters. For example, the HIC column may comprise two 0.2 micrometers (μm) filters upstream of the column. For example, the HIC column may comprise a 0.45/0.22 μm sized Polyvinylidene fluoride or, PVDF filter (30 inches cartridge), which may be subsequently referred to as the loading or, charge filter, followed by an additional 0.5/0.2 μm Polyethersulfone or, PES filter (30″ cartridge), which may be referred to as the column pre-filter. The loading or charge filter may be in-line for equilibration, loading or charging, and column washing only. With the exception of loading or charge, column wash, and sanitization, the pre-filter may be in-line whenever the column is in-line.

In some embodiments, the HIC column may comprise a resin with at least one hydrophobic ligand. For example, the resin for the HIC column may be chosen from alkyl-, aryl-ligands, and combinations thereof. For example, the HIC media may be chosen from butyl, hexyl, phenyl, octyl, and polypropylene glycol ligands. In some embodiments, the HIC column may comprise Capto phenyl as the hydrophobic ligand.

In some embodiments, the HIC unit operation may be performed using Capto Phenyl ImpRes resin generally packed to a bed height between 15 and 30 cm. The target bed height for the HIC column may be 21 cm. In some embodiments, the HIC column may have a bed height of 10 cm.

In some embodiments, the HIC column may have a column volume of 4.7 mL. In some embodiments, the HIC column resin may have a hydrophobicity from 45 to 50 min retention of lysozyme.

In some embodiments, the HIC column resin may have a flow velocity of up to 220 cm/h in a 1 m diameter column with a bed height of 20 cm at 20° C. that may be measured using process buffers with the same viscosity as water at 300 kPa.

In some embodiments, the HIC column resin may have a binding capacity of 19 mg bovine serum albumin (BSA)/mL of the solvent or the medium.

In some embodiments, the HIC column resin may have a pH stability of from pH 2 to 14. For example, the HIC resin may be stable in 1 M NaOH.

In some embodiments, the HIC column resin may be stored in 20% ethanol at 4° C. to 30° C.

In some embodiments, the HIC column may comprise a highly cross-linked agarose matrix with phenyl ligands. In some embodiments, the HIC column may comprise a matrix with an average particle size of 40 μm.

In some embodiments, the HIC column matrix may have a pH stability of from pH 2 to 14.

In some embodiments, a target flow rate of 120 cm/hr may be applied during the loading of dulaglutide composition in line with the buffer B solution onto the HIC column.

### Washing

As disclosed herein, methods of the present disclosure comprise washing the HIC column with a buffer mixture of a buffer A solution and the buffer B solution. In some embodiments, the buffer solution A may be Tris or tris(hydroxymethyl)aminomethane. For example, the buffer solution A may comprise 20 mM Tris. The conjugate acid of Tris has a partition coefficient (pKa) of 8.07 at 25° C., which implies that the buffer solution A may have an effective pH range between 7.1 and 9.1 (pKa±1) at room temperature.

The buffer mixture may comprise the same source for the buffer B solution as the in-line buffer B solution in the loading step. In some embodiments, the buffer mixture may comprise the buffer B solution from a different source but having the same concentration as the buffer B solution used in the loading step. As disclosed, the buffer solution B may comprise Tris and sodium sulfate salt. For example, the buffer solution B may comprise 20 mM Tris and 400 mM sodium sulfate for washing the HIC column.

The buffer mixture may comprise a salt chosen from ammonium sulfate, sodium sulfate, sodium chloride, ammonium chloride, sodium bromide, and a combination thereof. In some embodiments, the buffer mixture may comprise a sulfate salt, tris(hydroxymethyl)aminomethane, or a combination thereof. The sulfate salt may be chosen from ammonium sulfate and sodium sulfate. In various embodiments, the buffer mixture may comprise a cation chosen from Ba²⁺, Ca²⁺, Mg²⁺, Na⁺, K⁺, Rb⁺, and NH₄⁺, and/or an anion chosen from PO₄³⁻, SO₄²⁻, CH₂CO₃, Cl⁻, Br⁻, NO₃⁻, I⁻, and SCN⁻, and a combination thereof.

In some embodiments, the buffer A solution may comprise 20 mM Tris at a pH ranging from 7.7-8.3, and the buffer B solution may comprise 20 mM Tris, 400 mM sodium sulfate at a pH ranging from 7.7-8.3. Therefore, the buffer mixture may comprise 20 mM Tris, 320 mM sodium sulfate solution at a pH ranging from 7.7-8.3.

In some embodiments, the pH of the buffer mixture may be controlled throughout the process as a function of buffer composition and process step requirements and may be monitored as an integral aspect of the unit operations rather than as specific discrete in-process testing. pH control for viral inactivation prior to the anionic exchange chromatography unit operation may be controlled by at-line pH testing in the manufacturing facility.

In some embodiments, the buffer mixture for washing the HIC column may comprise from 15% to 25% of the buffer A solution and from 75% to 85% of the buffer B solution. For example, the buffer mixture may comprise 20% of the buffer A solution and 80% of the buffer B solution for washing the HIC column.

In some embodiments, washing of the HIC column may include using the buffer mixture at a linear rate ranging from 80 cm/hour to 160 cm/hour. In some embodiments, the linear rate may be 120 cm/hr.

In some embodiments, washing of the HIC column may use 2.7 column volumes (CV) to 3.3 CV of the buffer mixture. For example, washing of the HIC column may comprise use of 3.0 CV of the buffer mixture.

### Eluting

As disclosed herein, methods of the present disclosure comprise eluting dulaglutide from the HIC column using a linear gradient of a percentage of the buffer A and buffer B solutions and following the linear gradient with an isocratic hold. The isocratic hold may be based on a UV absorbance reading of an eluate from the HIC column. The method further comprises maintaining the isocratic hold until a front side cut is made and then dropping the percentage of the buffer B solution to 0% for dulaglutide to elute from the HIC column.

Loading of the dulaglutide composition in-line with the buffer B solution onto the HIC column results in the binding of dulaglutide to the HIC column. For example, dulaglutide may elute from the HIC column when the salt concentration in the buffer mixture is decreased in a linear or stepwise matter. In some embodiments, when washing the HIC column with buffer solutions, dulaglutide may remain bound to the HIC column while one or more impurities present in the composition may be washed with the buffer solutions. When initiating the elution phase, dulaglutide may start eluting from the HIC column.

As disclosed herein, eluting dulaglutide from the HIC column may be accomplished by a linear gradient step of a percentage of the buffer A and buffer B solutions, followed by an isocratic hold step, and by dropping the percentage of the buffer B solution to 0%. For example, the linear gradient decreases the percentage of the buffer B solution, while increasing the percentage of the buffer A solution. In some embodiments, the percentages of the buffer A and B solutions and the timing of the gradient is automated, e.g., based on several factors such as column volumes, UV signals, etc.

In some embodiments, eluting dulaglutide from the HIC column may comprise using the buffer B solution at a linear gradient ranging from 67.5% to 72.5% of the buffer B solution at a start setpoint and a linear gradient ranging from 5% to 15% of the buffer B solution at an end setpoint.

In some embodiments, eluting dulaglutide from the HIC column may comprise using the buffer A solution at a linear gradient ranging from 32.5% to 27.5% of the buffer A solution at a start setpoint and a linear gradient ranging from 95% to 85% of the buffer A solution at an end setpoint.

In some embodiments, a duration of the linear gradient duration varies based on the composition of the buffer A and buffer B solutions. In some embodiments, eluting dulaglutide with the linear gradient comprises 10 column volumes (CV)-12 CV of the buffer A and buffer B solutions. For example, eluting dulaglutide with the linear gradient may include 11.5 CV of the buffer A and buffer B solutions. In some embodiments, the linear gradient from 67.5% buffer B solution (320 mM sodium sulfate) to 10% buffer B solution (40 mM sodium sulfate) may be completed over 11.5 CV.

In some embodiments, eluting dulaglutide with the linear gradient may include a linear rate of 80 cm/hr to 160 cm/hr of the buffer mixture comprising the buffer A and buffer B solutions. In some embodiments, the linear flow rate of the buffer mixture for eluting dulaglutide from the HIC column may be 120 cm/hr.

As disclosed herein, eluting dulaglutide from the HIC column further comprises an isocratic hold step. In some embodiments, the isocratic hold may be based on a UV absorbance reading of an eluate from the HIC column. In some embodiments, the eluate may be a protein-containing material such as one or more impurities and/or dulaglutide depending on, e.g., the percentage of buffer A and buffer B solutions, the UV absorbance reading, etc. The UV absorbance reading may be measured at a wavelength of A 280 nanometers (nm).

In some embodiments, the isocratic hold may be accomplished with 10% of the buffer B solution. For example, the isocratic hold may be accomplished with 2.0 CV of 10% of the buffer B solution. The column volume of the buffer B solution used may indicate the duration of the isocratic hold. This may be built into the automation in the event that the front-side cut does not occur within the elution gradient. During elution, once a UV signal of 0.25 absorbance unit per centimeter (AU/cm) is reached, the linear gradient may then switch to isocratic conditions to maintain a constant concentration of salt. For example, once a UV signal of 0.25 AU/cm is reached, the linear gradient may then switch to isocratic conditions to maintain a constant concentration of sodium sulfate.

In some embodiments, the front side cut may be made after 1.5 CV of isocratic flow. A mainstream product collection step may begin after that. Dulaglutide may not elute from the HIC column or may remain bound to the HIC column resin during the washing step and may start eluting when the linear gradient or after the static hold when buffer B is at 0% is applied.

In some embodiments, the impurities in the dulaglutide composition may be removed or washed using the buffer mixture before the elution steps.

Impurities may be cleared on the front side of the mainstream and hence once the UV frontside cut target is achieved, the elution gradient may drop to 0% Buffer B to complete elution. In some embodiments, the target A 280 cut point for the backside of the mainstream may be 0.25 AU/cm. Following elution of the mainstream (i.e., dulaglutide), elution may be ended early by continuing to, e.g., a column regeneration step, as described below.

In some embodiments, the dulaglutide composition purification process may increase the concentration of dulaglutide initially present in the loading dulaglutide composition. For example, the dulaglutide composition purification process may increase the concentration of the dulaglutide to about 2.15 g/L to 6.51 g/L in the mainstream product after elution from the HIC column.

In some embodiments, the dulaglutide composition purification process may further reduce, e.g., a trace amount of impurities initially present in the loading of the dulaglutide composition. For example, the dulaglutide composition purification process may further reduce the concentration of the host cell proteins in the dulaglutide composition ranging from 30-70 ppm to about 10 ppm in the final mainstream. Further, for example, the dulaglutide composition purification process may reduce the concentration of the host cell proteins in the mainstream product after elution from the HIC column, e.g., the host cell proteins may range from 2 ppm to 11 ppm after elution from the HIC column.

In some embodiments, dulaglutide in the HIC product mainstream may be 0.2 μm filtered as an element of bioburden control prior to commencing the non-processing hold time (NPHT) at 2-8° C.

### Additional Steps

The disclosed method of purifying a dulaglutide composition according to one or more embodiments may include additional steps on demand. These steps may include equilibration of the column before the HIC unit operation, regeneration of the HIC column after use, sanitization of the HIC column before or after use, and storage of the HIC column after use. One or more of the additional steps may be performed in the methods of purifying a dulaglutide composition. Further, in some embodiments, the one or more additional steps may be performed in any combination.

### Equilibrating

In some embodiments, methods of the present disclosure further comprise, before loading the dulaglutide composition in-line with the buffer B solution onto the HIC column, equilibrating the HIC column in a downflow direction with 2 CV of a linear gradient at 32.5% of the buffer A and 67.5% of the buffer B solutions. For example, the HIC column may be equilibrated in a downflow direction with 32.5% of 20 mM Tris at pH around 8.0 and 67.5% of 20 mM Tris, 400 mM sodium sulfate at pH around 8.0. The equilibration of the HIC column may be generally performed in a downflow direction.

### Regenerating

In some embodiments, methods of the present disclosure further comprise, after elution is completed, regenerating or stripping the HIC column. For example, 3.0 CV of 0.01 N NaOH (diluted in-line from 0.1 N NaOH) may be applied to the HIC column in the upflow direction to regenerate the HIC resin. In some embodiments, the regeneration step may be conducted after each elution step to ensure efficient HIC column operation.

### Sanitizing

In some embodiments, methods of the present disclosure further comprise sanitizing the HIC column. For example, sanitizing comprises washing the HIC column in an upflow direction with 1.0 N NaOH with a target contact static hold time of not less than 30 minutes. For example, the HIC column may be sanitized with 2.0 CV of 1 N NaOH (diluted in-line from 3 N NaOH) followed by a target contact static hold time of not less than 30 minutes.

### Storing

In some embodiments, methods of the present disclosure further comprise two techniques for storing the HIC column after use depending on the time the HIC column is being held. For example, when the HIC column is held for more than 24 hours before loading the dulaglutide composition, storing the HIC column may comprise washing the HIC column with a storage buffer solution after use. In some embodiments, the storing buffer solution may be 0.01 N NaOH or an equivalent thereof. For example, storing the HIC column may comprise washing the HIC column with 2.0 CV of the storage buffer solution of 0.01 N NaOH in an upflow direction. In some embodiments, storing the HIC column may be performed for 2.0 CV in an upflow direction. In some embodiments, storing the HIC column may include a column storing solution of 0.01 N NaOH supplemented with 100 mM NaCl.

In other embodiments, when the HIC column is held for less than 24 hours before loading the dulaglutide composition, the HIC column may be re-equilibrated. For example, the equilibrating step described able is performed in order to store the HIC column for less than 24 hours before conducting another method for purifying a dulaglutide composition.

### EXAMPLES

The following examples serve only to illustrate the methods of the disclosure and are not intended to limit the scope of the disclosure, which is as set out in the claims.

### Example 1

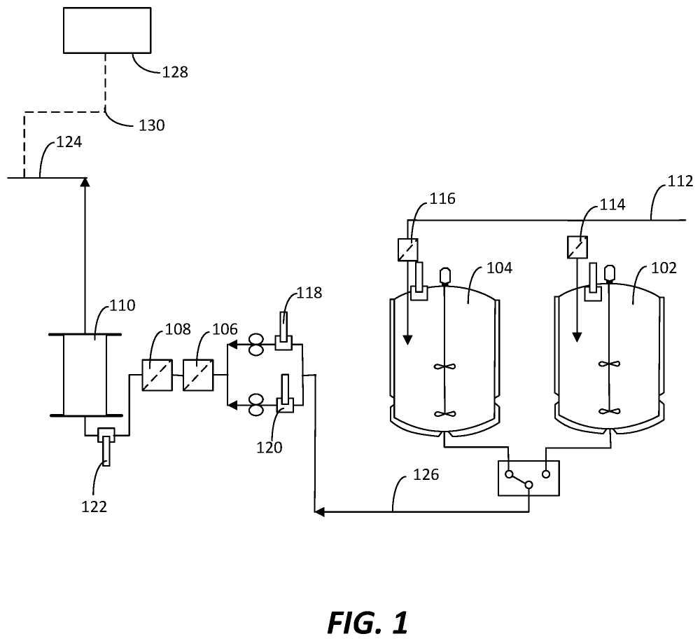

An example embodiment of the present disclosure is provided in Example 1 directed to a dulaglutide purification unit and a method for purifying a dulaglutide composition comprising a HIC column according to the present disclosure. For example, FIG. 1 illustrates a dulaglutide purification unit comprising hold vessels, a charge filter, a pre-filter column, a HIC column, in-lines, and outlines for processing the dulaglutide composition. The dulaglutide purification unit also includes flow rate controllers, pressure controllers, air sensors, pH sensors, temperature sensors, and pressure sensors. Describing the dulaglutide purification unit also illustrates a method performed according to the present disclosure.

In FIG. 1, the dulaglutide composition was loaded on the dulaglutide purification unit from a downstream product from a chromatography process, such as an anionic exchange chromatography unit. The composition was loaded onto the dulaglutide purification unit through a charge in-line 112 into two hold vessels 102 and 104. A first sensor 114 was positioned in line with the holding vessel 102 that was configured to measure, e.g., the temperature, pressure, agitation speed, and volume of the composition charged into the holding vessel 102.

Similarly, a second sensor 116 was positioned in line with the holding vessel 104 that was configured to measure, e.g., the temperature, pressure, agitation speed, and volume of the composition charged into the holding vessel 104. A mixed stream 126 from the hold vessels 102 and 104 was fed to the charge filter 106. Two sets of sensors 118 and 120 are positioned in parallel in line with the mixed stream 126 which was configured to measure the flow rate and pressure of the mixed stream 126.

After filtering the dulaglutide composition in the charge filter 106, the dulaglutide composition was filtered using a pre-column filter 108 prior to loading onto the HIC column 110. Another set of sensors 122 were positioned in line with the loading dulaglutide composition that was configured to measure the air, pH, temperature, and pressure of the dulaglutide composition for purification in the HIC column 110.

As disclosed, the HIC column was utilized to increase the overall purity of dulaglutide by increasing reversed phase main peak purity and by reducing HCP. A UV-spectroscopy unit 128 was connected to the HIC unit via a connector line 130 and was configured to measure the absorbance of the protein-containing material in the HIC product stream 124 at any given time. Based on the UV absorption readings, the HIC column operation parameters were automatically modified to capture dulaglutide and release one or more impurities eluting from the HIC column.

### Example 2

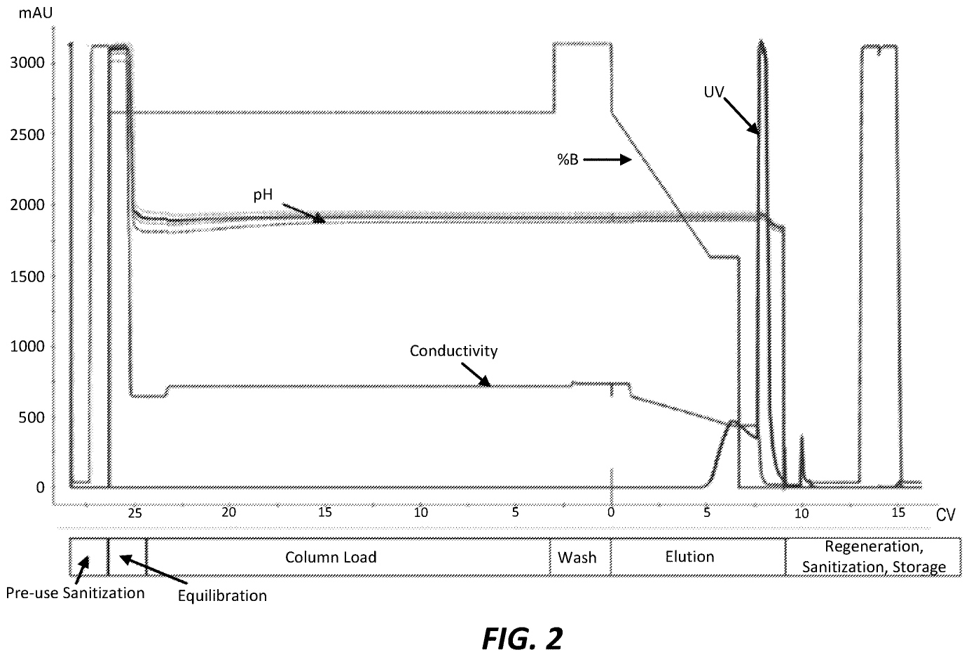

Examples of chromatograms generated during the above dulaglutide purifying unit and method as described under Example 1 is described in Example 2 and FIG. 2.

For example, Example 2 illustrates overlayed chromatograms during the HIC column operation for purifying a dulaglutide composition. As shown in FIG. 2, chromatograms were generated in several steps of the disclosed methods including sanitizing, equilibrating, column loading, washing, eluting, and regenerating, sanitizing and/or storing. FIG. 2 includes information regarding the UV-absorption of the dulaglutide composition as it was processed at different stages of the purification process using the dulaglutide purification operation using the HIC column. The chromatogram of FIG. 2 also includes data regarding the conductivity, pH, and % gradient of the buffer B solution.

### Buffer B Solution

As shown in FIG. 2, the HIC column was sanitized with about 2.0 column volumes (CV) of NaOH. Next, the HIC column was equilibrated with about 2.0 CV of 67.5% of the buffer B solution, followed by loading the column with the dulaglutide composition. The percentage of the buffer B solution in comparison to the buffer A solution may remain stable at about 67.5% of the B solution throughout the dulaglutide composition loading step.

Following the sanitizing and equilibrating steps, the HIC column was loaded and then washed with a buffer mixture of a buffer A solution and the buffer B solution, i.e., about 3.0 CV of 80% of the buffer B solution to remove residual components of the loading composition. After the washing step, eluting began at about 67.5% of the buffer B solution. The percentage (%) of the buffer B solution linearly dropped from 67.5% until a UV signal of 0.25 AU/cm was reached. During this linear gradient elution step, the percentage of the buffer A solution increased as the percentage of the buffer B solution decreased. The duration of the linear gradient of the elution step utilized about 5 CV of the buffer A and buffer B solutions.

During the linear gradient of the elution step, once a UV signal of 0.25 AU/cm was reached, the linear gradient switched to an isocratic hold maintaining a constant percentage of the buffer B solution. The isocratic hold step was maintained at about a 40% constant percentage of the buffer B solution and used about 1.5 CV of the buffer A and buffer B solutions, as shown in FIG. 2. Then, the front-side cut was made, and the mainstream collection began.

Impurities (e.g., protein-containing material) present in the loaded dulaglutide composition were cleared on the frontside of the mainstream and thus, once the UV frontside cut target was achieved, the elution gradient dropped to 0% of the buffer B solution to complete elution of dulaglutide, as shown in FIG. 2. Mainstream collection of the eluent containing dulaglutide continued until a UV signal of 0.25 AU/cm was reached, at which point a back-side cut was made to complete elution. After completing the elution, the final steps of regeneration, sanitization, and storage of the HIC column were conducted. In those steps of regeneration, sanitization and storage of the HIC column, the percentage of the buffer B solution remained 0%, as shown.

### pH and Conductivity

As shown in FIG. 2, after pre-use sanitization and equilibration of the HIC column, the pH of the analyzed solution remained stable around at 8.0 throughout the dulaglutide composition loading step, washing step, and elution step. As both the buffer A solution and buffer B solution were at pH 8.0, the variation in their concentration at different stages of these operations did not make any changes to the pH of the dulaglutide-containing analyzed solution.

### UV Absorbance

The UV absorbance of the analyzed solution at A 280 nm indicated the presence of dulaglutide and/or a protein-containing material in the analyzed solution. As illustrated, the chromatogram also shows that the UV absorbance of the analyzed solution at a wavelength of A 280 nm was close to zero in all the steps including pre-use sanitization, equilibration, column loading, washing, and the first phase of the elution step prior to the second phase of the elution step. This confirmed that dulaglutide or the protein-containing material, e.g., binds to the HIC column during the loading step and/or remains bound to the HIC column during the washing step and the first phase of the elution step. Thus, after 1.5 CV of isocratic flow, the front side cut was made, and mainstream collection began. Impurities or protein-containing material were cleared on the front side of the mainstream and thus, once the front side cut target was achieved, the elution gradient dropped to 0% of the buffer B solution to complete the elution of dulaglutide. The target A 280 cut point for the back side mainstream was 0.25 AU/cm.

At the end of the first phase of the elution step, referred to as the linear gradient period of the elution step and the beginning of the second phase of the elution step, referred to as the isocratic hold period of the elution step, the UV absorbance of the eluent at A 280 nm slowly increased. This indicated, e.g., that dulaglutide, a protein-containing material, eluted from the HIC column.

The UV-absorbance of the analyzed solution reached at a peak before rapidly dropping to a similar level as it was during the linear gradient step of elution or any prior steps. This sharp peak of the UV absorbance reading at A 280 nm also indicated the purity of the dulaglutide molecule or the protein-containing material in the analyzed solution.

### Example 3

Example 3 provides an exemplary selection of HIC column resins for use in methods of the present disclosure. Four different types of HIC column resins were tested in order to determine their effectiveness in purifying a dulaglutide composition using the methods disclosed herein. The four types of HIC column resins were Phenyl Sepharose HP (PSP), Butyl Sepharose HP (BSP), Capto Phenyl Impres (CPI), and Toyopearl Phenyl 650S resin (TPP).

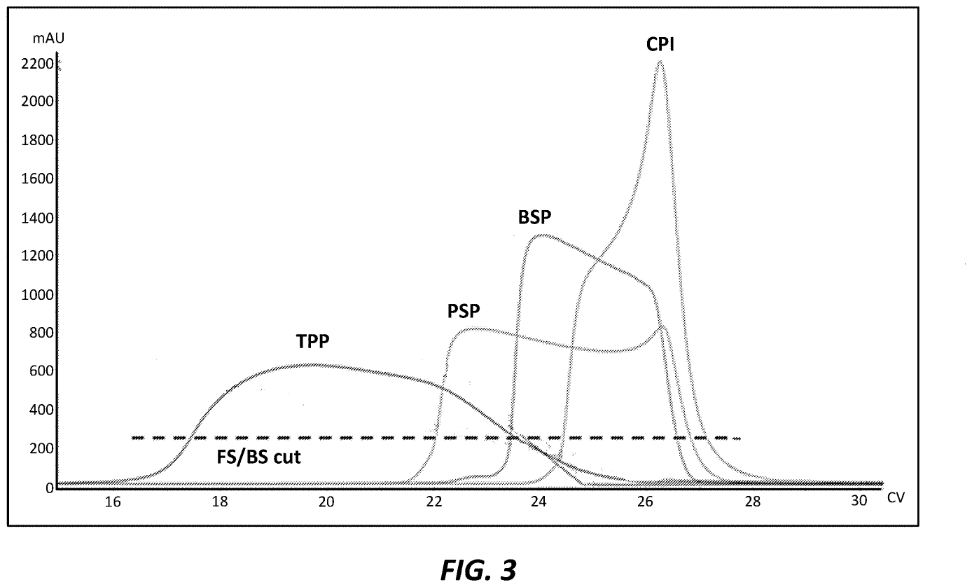

As shown in FIG. 3, a comparative study among these four different HIC column resin shows that the UV absorbance of the protein-containing materials at A 280 nm wavelength varied significantly although each of the test runs comprised the same dulaglutide composition load to begin the chromatography unit operation with. The intensity and sharpness of the peak absorbance indicated the % yield of dulaglutide. As shown in FIG. 3, the performance of the resins followed the following order: CPI>BSP>PSP>TPP. Table 1 below shows a summary of the % yield obtained by each of these HIC resins.

TABLE 1

| Resin Type | % Yield |
|---|---|
| Phenyl Sepharose HP (PSP) | 97.6 |
| Butyl Sepharose HP (BSP) | 95.4 |
| Capto Phenyl Impres (CPS) | 95.0 |
| Toyopearl Phenyl 650S resin (TPP) | 92.3 |

Other embodiments of the present disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of purifying a dulaglutide composition, the method comprising:
loading the dulaglutide composition in-line with a buffer B solution onto a hydrophobic interaction chromatography (HIC) column in a downflow direction, wherein dulaglutide from the dulaglutide composition binds to the HIC column;
washing the HIC column with a buffer mixture of a buffer A solution and the buffer B solution; and
eluting dulaglutide from the HIC column using a linear gradient of a percentage of the buffer A and buffer B solutions, wherein the linear gradient is followed by an isocratic hold based on a UV absorbance reading of an eluate from the HIC column indicating a protein-containing material is eluting from the HIC column, and maintaining the isocratic hold until a front side cut is made, and then dropping the buffer B solution to 0% for dulaglutide to elute from the HIC column.

2. The method of claim 1, wherein loading the dulaglutide composition with the buffer B solution is at a volume ratio ranging from 62.7% to 72.5% of the buffer B solution.

3. The method of claim 1, wherein loading the dulaglutide composition is at a target load ranging from 10 grams-20 grams of dulaglutide per liter of HIC resin.

4-39. (canceled)

40. A method of purifying a dulaglutide composition comprising a plurality of compounds from a fermentation process of mammalian cells, the method comprising:
loading the dulaglutide composition in-line with a buffer B solution at a volume ratio ranging from 62.7% to 72.5% and at a linear rate ranging from 80 cm/hr-160 cm/hr onto a hydrophobic interaction chromatography (HIC) column in a downflow direction, wherein dulaglutide from the dulaglutide composition binds to the HIC column;
washing the HIC column with a buffer mixture comprising from 15% to 25% of a buffer A solution and from 75% to 85% of the buffer B solution in the downflow direction at a linear rate ranging from 80 cm/hour-160 cm/hour for a duration of 2.7 column volumes (CV)-3.3 CV; and
eluting dulaglutide from the HIC column using a linear gradient comprising from 67.5% of the buffer B solution to 10% of the buffer B solution for a duration of 10 CV-12 CV, wherein the linear gradient is followed by an isocratic hold based on the UV absorbance reading of an eluate at A 280 nm with a range from 0.1 AU/cm to 0.5 AU/cm indicating a protein-containing material is eluting from the HIC column, maintaining the isocratic hold with a fixed % buffer B solution for 1.25-1.75 CV, making a front side cut with the buffer B solution at 0%, collecting a dulaglutide-containing eluate until a UV absorbance reading of the eluate at A 280 nm is within a range from 0.125 AU/cm to 0.25 AU, and making a back side cut when the UV absorbance reading of the eluate at A 280 nm is within 0.125 AU/cm to 0.25 AU.

41. The method of claim 40, wherein the HIC column comprises a highly cross-linked agarose matrix with phenyl ligands.

42. The method of claim 40, wherein the HIC column has a diameter ranging from 50 cm to 200 cm.

43-53. (canceled)
